(19) United States
(12) Patent Application Publication
(10) Pub. No.: US 2021/0225509 A1
(43) Pub. Date: Jul. 22, 2021

(54) METHOD, APPARATUS AND SYSTEM FOR GUIDING DIAGNOSIS-AND-TREATMENT, AND COMPUTER READABLE STORAGE MEDIUM

(71) Applicant: BOE TECHNOLOGY GROUP CO., LTD., Beijing (CN)

(72) Inventors: Xiaoyang WANG, Beijing (CN); Guangyu SHAO, Beijing (CN); Lei LV, Beijing (CN); Wenjuan LI, Beijing (CN); Chaozheng LIU, Beijing (CN); Jiankun ZHANG, Beijing (CN); Haifeng LIU, Beijing (CN); Rong DAI, Beijing (CN); Dachuan WANG, Beijing (CN)

(73) Assignee: BOE TECHNOLOGY GROUP CO., LTD., Beijing (CN)

(21) Appl. No.: 16/975,505

(22) PCT Filed: Jan. 8, 2020
(86) PCT No.: PCT/CN2020/070883

§ 371 Date: Aug. 25, 2020

(30) Foreign Application Priority Data
Mar. 15, 2019 (CN) 201910197463.4

(51) Int. Cl. G16H 50/20; G16H 15/00; G16H 10/60; G16H 40/20; G16H 70/20; G06F 3/048; H04L 29/08

(52) U.S. Cl. CPC G16H 50/20; G16H 15/00; G16H 10/60; H04L 67/22; G16H 70/20; G06F 3/048; G16H 40/20

(57) ABSTRACT

The present disclosure provides a method, apparatus and system for guiding diagnosis-and-treatment, and a computer readable storage medium. The method for guiding diagnosis-and-treatment includes the following steps: acquiring basic process information of the diagnosis-and-treatment; determining flow progress information; and generating a diagnosis-and-treatment flowchart and corresponding status indication information according to the basic flow information and the flow progress information, wherein the status indication information indicates a status progress of at least one flow-stage in the diagnosis-and-treatment flowchart.

### CROSS-REFERENCE OF RELATED APPLICATIONS

This application claims priority to Chinese Patent Application No. 201910197463.4 filed on Mar. 15, 2019, which is incorporated by reference as a part of this application.

### TECHNICAL FIELD

The present disclosure relates to the technical field of Internet communication, in particular to a method, an apparatus and a system for guiding diagnosis-and-treatment, and a computer readable storage medium.

### BACKGROUND

With the gradual development of computer and Internet communication technology, intelligent interactive systems for medical field are constantly improving, and some of the diagnosis-and-treatment functions are gradually realized on Internet interfaces or mobile terminals.

However, at present, the realization of some of the diagnosis-and-treatment functions in the medical field is relatively isolated from each other, and operations are relatively cumbersome. For example, on the Internet or at the mobile terminals, an independent registration function can be used to make an appointment registration, and an independent payment function can also be used to scan a code for payment. However, a customized and differentiated service of the whole flow of the diagnosis-and-treatment can't be realized according to requirements such as different patients, different hospitals and different diseases, so there still exist a lot of inconvenience for the patients.

### SUMMARY

One aspect of the present disclosure provides a method for guiding diagnosis-and-treatment, which comprises the following steps: acquiring basic process information of the diagnosis-and-treatment; determining flow progress information; and generating a diagnosis-and-treatment flowchart and corresponding status indication information according to the basic flow information and the flow progress information, wherein the status indication information indicates a status progress of at least one flow-stage in the diagnosis-and-treatment flowchart.

According to an embodiment of the present disclosure, the method further comprises the following steps: acquiring flow-stage inquiry information; and presenting, according to the flow-stage inquiry information, specific contents of a flow-stage in the diagnosis-and-treatment flowchart corresponding to the flow-stage inquiry information.

According to an embodiment of the present disclosure, the method further comprises: when presenting the specific contents of the flow-stage in the diagnosis-and-treatment flowchart, further presenting an interactive option for the diagnosis-and-treatment flowchart, wherein the diagnosis-and-treatment flowchart is presented when an interactive instruction is acquired through the interactive option.

According to an embodiment of the present disclosure, the method further comprises the following steps: acquiring interaction history information; and generating a corresponding historical diagnosis-and-treatment flowchart according to the interaction history information.

According to an embodiment of the present disclosure, determining the flow progress information further comprises acquiring the flow progress information updated in real time through a server.

According to another aspect of the present disclosure, there is also provided an apparatus for guiding diagnosis-and-treatment, which comprises: an acquisition unit configured to acquire basic flow information of the diagnosis-and-treatment; a determination unit configured to determine flow progress information; and a generation unit configured to generate a diagnosis-and-treatment flowchart and corresponding status indication information according to the basic flow information and the flow progress information, wherein the status indication information indicates a status progress of at least one flow-stage in the diagnosis-and-treatment flowchart.

According to an embodiment of the present disclosure, the generation unit acquires flow-stage inquiry information; and present, according to the flow-stage inquiry information, specific contents of a flow-stage in the diagnosis-and-treatment flowchart corresponding to the flow-stage inquiry information.

According to an embodiment of the present disclosure, when the generation unit presents the specific contents of the flow-stage in the diagnosis-and-treatment flowchart, the generation unit further present an interactive option for the diagnosis-and-treatment flowchart, wherein the diagnosis-and-treatment flowchart is presented when an interactive instruction is acquired through the interactive option.

According to an embodiment of the present disclosure, the acquisition unit acquires interaction history information; and the generation unit generates a corresponding historical diagnosis-and-treatment flowchart according to the interaction history information.

According to the embodiment of the present disclosure, the determination unit acquires the flow progress information updated in real time through a server.

According to another aspect of the present disclosure, there is also provided an apparatus for guiding diagnosis-and-treatment, which comprises one or more processors and one or more memories, wherein the memories store computer readable codes which, when executed by the one or more processors, perform the following steps: acquiring basic process information of the diagnosis-and-treatment; determining flow progress information; and generating a diagnosis-and-treatment flowchart and corresponding status indication information according to the basic flow information and the flow progress information, wherein the status indication information indicates a status progress of at least one flow-stage in the diagnosis-and-treatment flowchart.

According to another aspect of the present disclosure, there is also provided a computer-readable storage medium on which computer instructions are stored, wherein when the computer instructions are executed on a processor, the processor is caused to perform the following steps: acquiring basic process information of the diagnosis-and-treatment; determining flow progress information; and generating a diagnosis-and-treatment flowchart and corresponding status indication information according to the basic flow information and the flow progress information, wherein the status indication information indicates a status progress of at least one flow-stage in the diagnosis-and-treatment flowchart.

According to another aspect of the present disclosure, there is also provided a system for guiding diagnosis-and-treatment, which comprises an interactive platform configured to execute the method for the diagnosis-and-treatment as previously described; a hospital management system; and a server, wherein the server communicates and interacts with the interactive platform and the hospital management system respectively.

### BRIEF DESCRIPTION OF THE DRAWINGS

In order to more clearly describe embodiments of the present disclosure or technical solutions known in the prior art, drawings needed in the description of the embodiments or the prior art will be briefly introduced in the following. Obviously, the drawings in the following description are only some embodiments of the present disclosure, and for those skilled in the art, other drawings can be obtained according to these drawings without inventive labor.

FIG. 1 illustrates a method for guiding a diagnosis-and-treatment according to an embodiment of the present disclosure;

FIG. 2 illustrates an example of displaying a user interface of a diagnosis-and-treatment flowchart according to an embodiment of the present disclosure;

FIG. 3 illustrates a further exemplary implementation of the method for guiding the diagnosis-and-treatment according to an embodiment of the present disclosure;

FIG. 4 illustrates a further exemplary implementation of the method for guiding the diagnosis-and-treatment according to an embodiment of the present disclosure;

FIG. 5 illustrates an example of a sidebar interaction option according to an embodiment of the present disclosure;

FIG. 6 illustrates a further exemplary implementation of the method for guiding the diagnosis-and-treatment according to an embodiment of the present disclosure;

FIG. 7 illustrates a structural block diagram of an apparatus for guiding a diagnosis-and-treatment according to an embodiment of the present disclosure;

FIG. 8 illustrates a structural block diagram of the apparatus for guiding the diagnosis-and-treatment according to an embodiment of the present disclosure;

FIG. 9 illustrates a structural block diagram of an example of a system for guiding a diagnosis-and-treatment according to an embodiment of the present disclosure.

### DETAILED DESCRIPTION

In the following, the technical solution in the embodiments of the present disclosure will be clearly and completely described with reference to the drawings in the embodiments of the present disclosure. Obviously, the described embodiments are only some embodiments of the present disclosure, not all embodiments. Based on the embodiments in the present disclosure, all other embodiments obtained by those of ordinary skill in the art without inventive labor are within the protection scope of the present disclosure.

As used in this disclosure, “first”, “second” and similar words do not indicate any order, quantity or importance, but are only used to distinguish different components. Similarly, similar words such as “include” or “comprise” mean that the elements or objects appearing before the word cover the listed elements or objects and their equivalents appearing after the word, but do not exclude other elements or objects. Similar words such as “connected to” or “connected with” are not limited to physical or mechanical connections, but can include electrical connections, whether direct or indirect.

Flowcharts are used in this disclosure to illustrate steps of the methods according to the embodiments of the present disclosure. It should be understood that the preceding or following steps are not necessarily performed accurately in sequence. On the contrary, the various steps can be processed in reverse order or simultaneously. At the same time, other operations can be added to these processes, or one step or several steps can be removed from these processes.

An intelligent interactive system aiming at the medical field may comprises the following parts of: a Hospital Information System (HIS), a laboratory information management system (LIS), a Picture Archiving and Communication Systems (PACS), a transmission system and doctor workstation, etc., so as to realize the collection, storage, processing, extraction and data exchange of diagnosis-and-treatment information.

In the current era of mobile Internet, information technology, as a main driving force, makes social productive forces continuously develop in width and depth. The medical field has also been applied more and more on mobile terminals. However, on the one hand, implementation methods of each part of the diagnosis-and-treatment functions are relatively isolated and the operating methods thereof are different, which will bring the disadvantages of high learning cost and inconvenient use. On the other hand, the diagnosis-and-treatment flow itself is personalized, and the diversity of disease types, the difference of the target objects, the difference between an initial visit and a follow-up visit, etc., will lead to a large or small difference in each diagnosis-and-treatment flow, which will further increase the inconvenience of using the above applications.

The embodiments of the present disclosure provide a method and apparatus that can realize the ordering and status indication of the whole diagnosis-and-treatment process, so as to improve the user experience and improve the efficiency of the diagnosis-and-treatment.

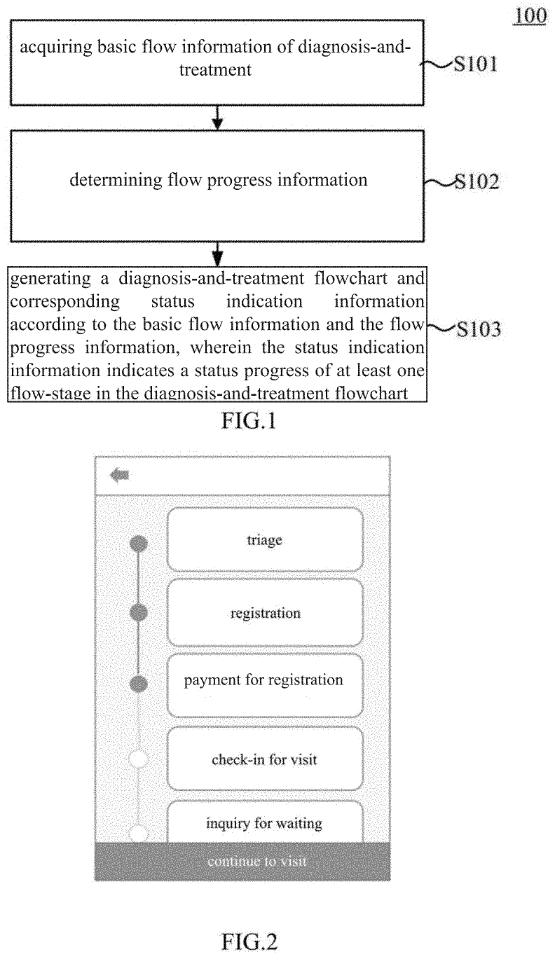

Next, a method 100 for guiding diagnosis-and-treatment according to an embodiment of the present disclosure will be described with reference to FIG. 1. FIG. 1 illustrates a method for guiding a diagnosis-and-treatment according to an embodiment of the present disclosure.

First, according to step S101, basic flow information of the diagnosis-and-treatment is obtained.

According to the embodiments of the present disclosure, in this step, the acquired basic flow information of the diagnosis-and-treatment may include all available diagnosis-and-treatment flow-stages in the diagnosis-and-treatment process applicable to the method, apparatus and system for guiding the diagnosis-and-treatment according to the embodiments of the present disclosure.

In an example, the basic flow information of the diagnosis-and-treatment may include information on various flow-stages such as triage, registration, payment for registration, check-in for visit, inquiry for waiting, self-service payment, inquiry for guidance note, display of electronic report, satisfaction survey, etc. In the embodiments of the present disclosure, the information for each flow-stage in the basic flow information can realize different functions correspondingly, and can further display or prompt corresponding information for the target object visiting a doctor.

Among them, in the flow-stage of the triage, personal information, hospital and department information, information on symptoms of disease and other contents of the target object can be acquired to help the target object locating the disease and determining triage information. Optionally, in the flow-stage of the triage, by acquiring the personal information such as age and gender of the target object, the location or selected hospital of the target object, the information on symptoms of disease such as location or type of pain, information on whether it is an initial visit or a follow-up visit, etc., the disease type can be determined or advices on the visit and corresponding triage information (such as information of the hospital department to which the target object needs to register, or even specific doctors) are provided so as to guide the target object to perform the subsequent flow-stage of the registration. Optionally, the information on symptoms of disease can also be acquired by a physiological information measurement apparatus. For example, in the flow-stage of the triage, corresponding physiological information data of the target object can be acquired through various sensors in the physiological information measurement apparatus, and once the acquired physiological information data is abnormal, the target object can be prompted, or a triage function can be automatically initiated immediately according to triage information corresponding to the abnormal physiological information data, and even the appointment registration in the nearest hospital can be facilitated.

In the flow-stage of the registration, the target object can be guided to make the registration according to the selection of the target object or the previously determined triage information based on scheduling situations of doctors in different hospitals, different departments and different times. Among them, by acquiring the registration information of the target object, such as name, contact information, date for visit, medical insurance type, medical insurance card number, registered hospital and department, specific registration information of the target object can be generated and the target object is guided to perform the registration. The above flow-stage of the registration can be applied to the functions of the appointment registration, instant registration, registration on current day and so on.

After the target object completes the registration, payment can be made through the flow-stage of the payment for registration. Specific payment manners may include online payment (credit card, WeChat, Alipay, etc.), or counter payment by showing corresponding registration information, etc., which is not limited here.

After the flow-stage of the payment for registration is completed, the function of the check-in for visit can be implemented in the flow of the check-in for visit. For example, when there is registration information of the target object within a preset time period or on current day, corresponding information for the check-in for visit (such as appointment number, QR code, bar code, etc.) can be generated and provided to the target object, so that the target object can check-in for visit through a self-service check-in machine or a triage desk. In the flow-stage of the check-in for visit, corresponding displayed contents can include, for example, checked-in status and non-checked-in status.

After the flow-stage of the check-in for visit is completed, information on the checked-in status can be displayed first, and then in the subsequent flow-stage of the inquiry for waiting, the target object can be prompted in various manners to enter a waiting queue, and/or by displaying current waiting status, waiting department and doctor information. When the target object has completed or is about to complete the waiting in the waiting queue, a prompt for the waiting may further be generated, and more detailed information on the visit can be prompted, such as the floor for the visit, the room number of the doctor, etc. Optionally, in this flow-stage, manners for prompting such us short messages, sounds, or vibrations, etc. can be used for prompting the waiting status, so as to prevent the target object from missing the time for the visit.

After the target object completes the visit, and the doctor completes the diagnosis for the target object, the doctor may provide corresponding diagnosis results, drug list, etc., or may require the target object to do a further examination and test for definite diagnosis. At this time, the subsequent flow-stage of the self-service payment, flow-stage of the inquiry for guidance note, or flow-stage of the display of electronic report, etc. may be involved. Among them, when the target object needs to get medicine or perform a further examination and test according to the doctor's diagnosis, it is possible to pay for the medicine or the examination and test through the flow of the self-service payment, and the payment manners can also include online payment (credit card, WeChat, Alipay, etc.), or counter payment by showing personal information, registration information, corresponding information of the medicine or examination and test, etc., which are not limited here. After the payment is completed, a guidance note can be generated in the flow-stage of the inquiry for the guidance note. Optionally, the guidance note can provide information for getting medicine (name of the medicine, place for getting the medicine, etc.) and information on items of examination (contents of the examination, department and place of the examination, etc.), so that the target object can conveniently go to corresponding place to complete various required operations according to the guidance note. Optionally, the flow-stage of the inquiry for the guidance note can also prompt the target object according to the address, time and order requirements of different operations, so as to facilitate the target object to arrange the time and items of the examination reasonably, and to get medicine and wait in line for the items of the examination accordingly. For example, when the target object is waiting in line for a blood draw, and receives a reminder of B-ultrasound examination at this time, the target object can choose to return to B-ultrasound department first, or choose to continue to complete the blood draw and to wait in line for the B-ultrasound examination again, therefore the visit time of each target object is saved, and the hospital can be facilitated to make reasonable diversion.

Then, in the flow-stage of the display of electronic report, the target object can view the electronic report such as the diagnosis result made by the doctor, a drug list, an examination report of various examinations, a hospitalization report, etc. In addition, in this flow-stage, targeted contents such as advices for a follow-up visit and precautions are also recommended according to the electronic report of the target object.

After the completion of the whole flow of the diagnosis-and-treatment as described above, the target object can further enter the flow-stage of the satisfaction survey according to the visit experience of the whole flow, so as to feedback the satisfaction of the visit and promote the continuous improvement of the hospital and all parties providing services.

Examples of various basic flow information of the diagnosis-and-treatment according to embodiments of the present disclosure have been described in detail above. In particular applications of the embodiments of the present disclosure, the basic flow information may be different according to the requirements of different target objects, different diseases and different hospital departments, and is not limited here.

At step S102, flow progress information is determined.

In the embodiments of the present disclosure, the flow progress information may be different according to the visit flow-stage which the target object visiting a doctor is currently undergoing. For example, when the target object is in the flow-stage of triage, the flow progress information can be, for example, information on symptoms of disease for the target object, etc. When the target object is in the flow-stage of the payment for registration or in the flow-stage of the check-in for visit, the flow progress information can be, for example, whether the payment for registration or the check-in for visit is completed. When the target object is in the flow-stage of the inquiry for guidance note, the flow progress information can be, for example, the completed tasks of the guidance note and the uncompleted tasks of the guidance note for the target object, or specific contents of the examination report with the examination results having been obtained. When the target object has completed all flow-stages of the diagnosis-and-treatment, the flow progress information can enter the flow-stage of the satisfaction survey, etc.

Optionally, determining the flow progress information may further comprises acquiring the flow progress information updated in real time through a server. The server in the embodiments of the present disclosure may be the aforementioned Hospital Information System (HIS), laboratory information management system (LIS), picture archiving and communication systems (PACS), or contain the contents stored and interacted in these systems. By interacting with the server, the flow progress information can be updated in real time, and the diagnosis-and-treatment flowchart and the state progresses of the flow-stages in the diagnosis-and-treatment flowchart accordingly.

In step S103, a diagnosis-and-treatment flowchart and corresponding status indication information are generated according to the basic flow information and the flow progress information, wherein the status indication information indicates a status progress of at least one flow-stage in the diagnosis-and-treatment flowchart.

According to the embodiments of the present disclosure, a whole diagnosis-and-treatment flowchart for the target object can be generated according to the basic flow information and the flow progress information, and a state indication can be performed for the state progress of one or more flow-stages in the whole diagnosis-and-treatment flowchart. Considering factors such as diversity of diseases, the difference between the situations of target objects visiting a doctor, etc. as mentioned above, the whole diagnosis-and-treatment flowchart for different target objects may be significantly different, and the diagnosis-and-treatment flowchart may also be different according to different progresses of the visit of a target object. For example, if the target object is in the stage of a follow-up visit, a certain target department or doctor may have been determined already, and the target object may even visit directly without registration. At this time, the flow-stage of the triage and the flow-stage of the registration may be omitted. For another example, when the doctor can directly provide diagnosis results after diagnosis and the aforementioned operation of getting medicine or further examination is no longer needed, the diagnosis-and-treatment flow of the target object may directly jump from the flow-stage of the check-in for visit to the display of electronic report flow as required, without performing the flow-stage of the self-service payment, the flow-stage of the inquiry for guidance note, etc. Or, for example, when the target object has undergone corresponding examinations in the flow-stage of the inquiry for the guidance note and needs to register again to check results, the diagnosis-and-treatment flow of the target object may be necessary to jump to the flow-stage of the registration again. Therefore, in view of the above-mentioned various personalized situations of the visit, according to the embodiments of the present disclosure, the diagnosis-and-treatment flowchart can be generated for different target objects, different diseases and different flow progresses, and the status progress can be constantly changed and updated according to actual situations.

Optionally, the status progress of the flow according to the embodiments of the present disclosure can be displayed through different status indication information. For example, in the diagnosis-and-treatment flowchart, the completed flow-stage can be displayed in gray, while the uncompleted flow-stage can be displayed in white. Accordingly, for the completed flow-stage, specific contents of the flow-stage according to the selection of the target object can be provided, while for the uncompleted flow-stage, the specific contents of the flow-stage cannot be displayed, or only some operation prompts of the flow-stage can be displayed.

FIG. 2 illustrates an example of displaying a user interface of a diagnosis-and-treatment flowchart according to an embodiment of the present disclosure. It can be inferred from the diagnosis-and-treatment flowchart shown in FIG. 2 that in this example, the flow-stage of triage and the flow-stage of the registration and the flow-stage of the payment for registration have all been completed, and what is needed to be performed currently is the subsequent flow-stage of the check-in for visit.

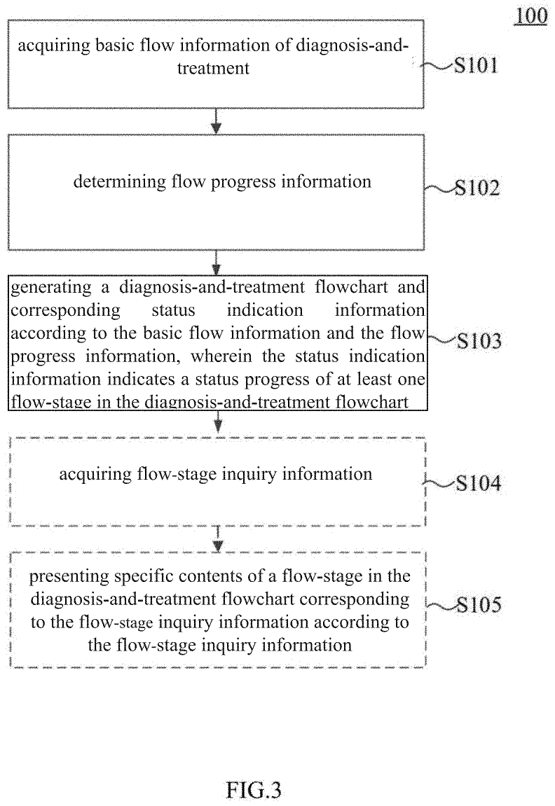

FIG. 3 illustrates a further exemplary implementation of the method for guiding the diagnosis-and-treatment according to an embodiment of the present disclosure. As shown in FIG. 3, the method for guiding the diagnosis-and-treatment according to the embodiments of the present disclosure may further comprise: acquiring flow-stage inquiry information (step S104), and presenting specific contents of a flow-stage in the diagnosis-and-treatment flowchart corresponding to the flow-stage inquiry information according to the flow-stage inquiry information (step S105). Optionally, the flow-stage inquiry information according to the embodiments of the present disclosure may be acquired by selecting a corresponding flow on the user interface by the target object. For example, the flow-stage inquiry information may be obtained by clicking a certain flow-stage in the diagnosis-and-treatment flowchart by the target object. In addition, optionally, specific contents of the flow-stage presented by the flow-stage inquiry information may be different with different flow-stages. For example, the specific contents of the flow-stage of the triage can be triage information, the specific contents of the flow-stage of the registration can be registration information, the specific contents of the flow-stage of the payment for registration can be whether the payment for registration is made, the specific contents of the flow-stage of the check-in for visit can be whether the check-in is made, the specific contents of the flow-stage of the waiting can be waiting order and department information, the specific contents of the flow-stage of the self-service payment can be whether the payment is made, the specific contents of the flow-stage of the inquiry for guidance note can be a guidance note, a drug list, whether an examination is to be made, etc., the specific contents of the flow-stage of the display of the electronic report can be an electronic report, and the specific contents of the flow-stage of the satisfaction survey can be specific options for satisfaction survey. In addition, as mentioned above, for the completed flow-stage, specific contents of the flow-stage can be provided, while for the uncompleted flow-stage, specific contents of the flow-stage cannot be provided, or only operation prompts of the flow-stage can be provided.

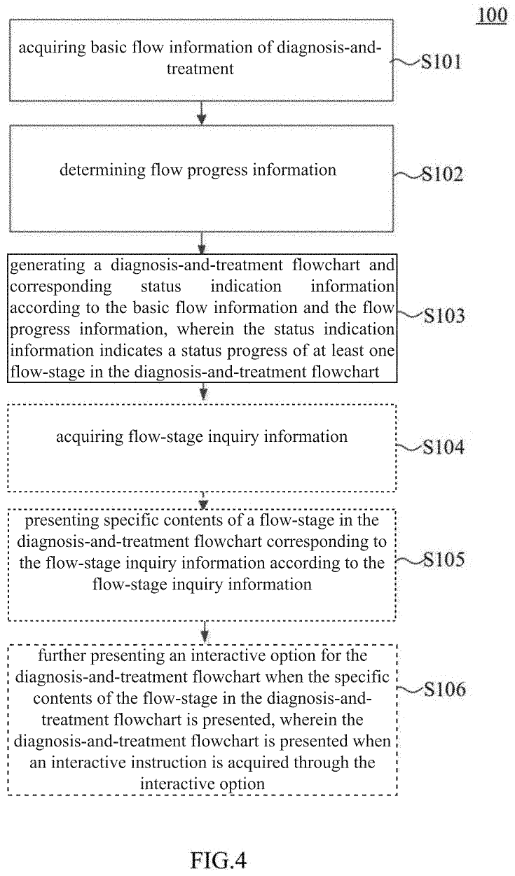

Further, FIG. 4 illustrates a further exemplary implementation of the method for guiding the diagnosis-and-treatment according to an embodiment of the present disclosure. As shown in FIG. 4, the method for guiding the diagnosis-and-treatment according to the embodiments of the present disclosure may further comprise: when presenting the specific contents of the flow-stage in the diagnosis-and-treatment flowchart, further presenting an interactive option for the diagnosis-and-treatment flowchart (step S106), wherein the diagnosis-and-treatment flowchart is presented when an interactive instruction is acquired through the interactive option. Specifically, after presenting the specific contents of a corresponding flow-stage by obtaining the flow-stage inquiry information, the whole diagnosis-and-treatment flowchart can be further presented on the basis of the specific contents of a certain flow-stage through the interactive option for the whole diagnosis-and-treatment flowchart provided in this step. For example, the interactive option can be a sidebar button on the presentation interface of the specific contents of a certain process, and the sidebar button can be fixed or suspended, which is not limited here. The target object can provide an interactive instruction by clicking the sidebar button. After the interactive instruction provided by the target object is acquired, the diagnosis-and-treatment flowchart for the target object can be displayed on the sidebar of the user interface according to the interactive instruction of the target object. Optionally, the diagnosis-and-treatment flowchart presented in the sidebar may also prompt the flow-stage inquiry information when the target object clicks a corresponding flow-stage, and jump to the specific contents of the flow-stage corresponding to the flow-stage inquiry information again according to situations, so as to realize the display of the whole flow of the diagnosis-and-treatment and the switching and jumping of the displayed contents among different flow-stages. FIG. 5 illustrates an example of a sidebar interaction option according to an embodiment of the present disclosure. As shown in FIG. 5, on the premise of displaying the specific contents of a certain flow-stage, the diagnosis-and-treatment flowchart of the target object can be displayed on the sidebar for the target object to click to select a corresponding flow-stage.

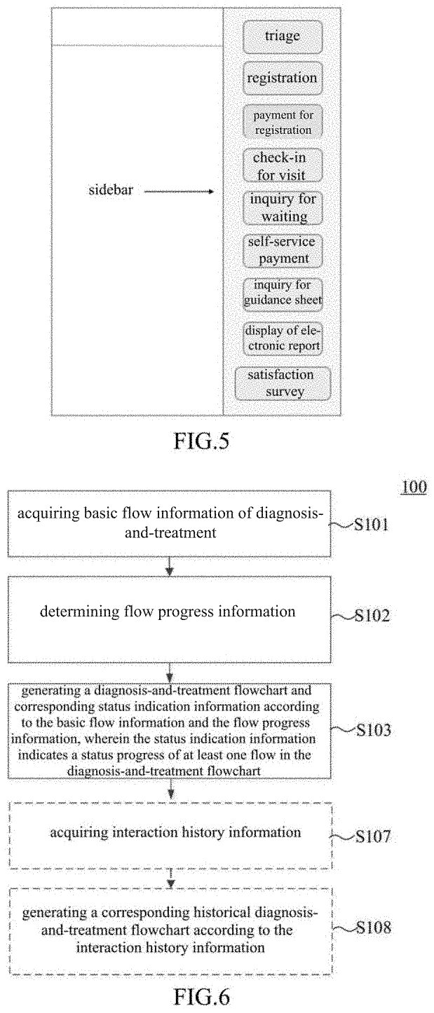

FIG. 6 illustrates a further exemplary implementation of the method for guiding the diagnosis-and-treatment according to an embodiment of the present disclosure. As shown in FIG. 6, the method for guiding the diagnosis-and-treatment according to the embodiments of the present disclosure may further comprise: acquiring interaction history information (step S107); and generating a corresponding historical diagnosis-and-treatment flowchart according to the interaction history information (step S108). Historical visit information of the target object provides a very important reference for the triage and visit of the target object. Therefore, in the embodiments of the present disclosure, historical diagnosis-and-treatment flowchart can be presented by obtaining the interaction history information, and a corresponding electronic report and other contents can be displayed, as a reference for the target object or the doctor.

The method for guiding the diagnosis-and-treatment according to the embodiments of the present disclosure can realize flow customization and status indication aiming at different diagnosis-and-treatment target objects and different diagnosis-and-treatment flows can be realized, so as to improve user experience and improve the efficiency of visit.

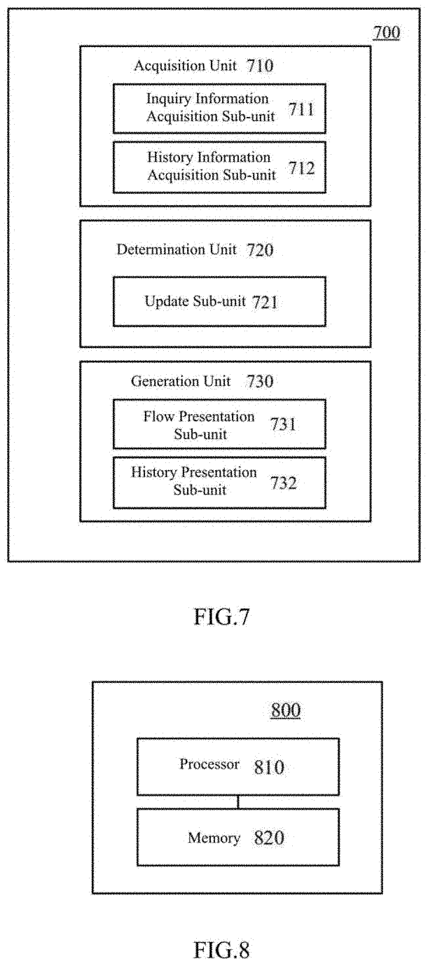

Next, an apparatus 700 for guiding a diagnosis-and-treatment according to an embodiment of the present disclosure will be described with reference to FIG. 7.

As shown in FIG. 7, the apparatus 700 for guiding a diagnosis-and-treatment according to an embodiment of the present disclosure may include an acquisition unit 710, a determination unit 720, and a generation unit 730.

The acquisition unit 710 is configured to acquire basic flow information of the diagnosis-and-treatment.

According to the embodiments of the present disclosure, the acquired basic flow information of the diagnosis-and-treatment may include all available diagnosis-and-treatment flow-stages in the diagnosis-and-treatment process applicable to the apparatus and system for guiding the diagnosis-and-treatment guiding according to the embodiments of the present disclosure.

In an example, the basic flow information of the diagnosis-and-treatment may include information on various flow-stages such as triage, registration, payment for registration, check-in for visit, inquiry for waiting, self-service payment, inquiry for guidance note, display of electronic report, satisfaction survey, etc. In the embodiments of the present disclosure, the information for each flow-stage in the basic flow information can realize different functions correspondingly, and can further display or prompt corresponding information for the target object visiting a doctor.

Among them, in the flow-stage of the triage, personal information, hospital and department information, information on symptoms of disease and other contents of the target object can be acquired to help the target object locating the disease and determining triage information. Optionally, in the flow-stage of triage, by acquiring the personal information such as age and gender of the target object, the location or selected hospital of the target object, the information on symptoms of disease such as location or type of pain, information on whether it is an initial visit or a follow-up visit, etc., the disease type can be determined or advices on the visit and corresponding triage information (such as information of the hospital department to which the target object needs to register, or even specific doctors) are provided so as to guide the target object to perform the subsequent flow-stage of the registration. Optionally, the information on symptoms of disease can also be acquired by a physiological information measurement apparatus. For example, in the flow-stage of the triage, corresponding physiological information data of the target object can be acquired through various sensors in the physiological information measurement apparatus, and once the acquired physiological information data is abnormal, the target object can be prompted, or a triage function can be automatically initiated immediately according to triage information corresponding to the abnormal physiological information data, and even the appointment registration in the nearest hospital can be facilitated.

In the flow-stage of the registration, the target object can be guided to make the registration according to the selection of the target object or the previously determined triage information based on scheduling situations of doctors in different hospitals, different departments and different times. Among them, by acquiring the registration information of the target object, such as name, contact information, date for visit, medical insurance type, medical insurance card number, registered hospital and department, specific registration information of the target object can be generated and the target object is guided to perform the registration. The above flow of the registration can be applied to the functions of the appointment registration, instant registration, registration on current day and so on.

After the target object completes the registration, payment can be made through the flow-stage of the payment for registration. Specific payment manners can include online payment (credit card, WeChat, Alipay, etc.), or counter payment by showing corresponding registration information, etc., which is not limited here.

After the flow-stage of the payment for registration is completed, the function of the check-in for visit can be implemented in the flow-stage of the check-in for visit. For example, when the target object has registration information within a preset time period or on current day, corresponding registration information (such as appointment number, two-dimensional code, bar code, etc.) can be generated and provided to the target object, so that the target object can check-in for visit through a self-service check-in machine or a triage desk. In the flow of the check-in for visit, corresponding displayed contents can include, for example, checked-in status and non-checked-in status.

After the flow-stage of the check-in for visit is completed, information on the checked-in status can be displayed first, and then in the subsequent flow-stage of the inquiry for waiting, the target object can be prompted in various manners to enter a waiting queue and/or by displaying current waiting status, waiting department and doctor information. When the target object has completed or is about to complete the waiting in the waiting queue, a prompt for the waiting may further be generated, and more detailed information on the visit can be prompted, such as the floor for the visit, the room number of the doctor, etc. Optionally, in this flow-stage, manners for prompting such us short messages, sounds, or vibrations, etc. can be used for prompting the waiting status, so as to prevent the target object from missing the time for the visit.

After the target object completes the visit, and the doctor completes the diagnosis for the target object, the doctor may provide corresponding diagnosis results, drug list, etc., or may require the target object to do a further examination and test for definite diagnosis. At this time, the subsequent flow-stage of the self-service payment, flow-stage of the inquiry for guidance note or flow-stage of the display of electronic report, etc. may be involved. Among them, when the target object needs to get medicine or perform a further examination and test according to the doctor's diagnosis, it is possible to pay for the medicine or the examination and test through the flow-stage of the self-service payment, and the payment manners can also include online payment (credit card, WeChat, Alipay, etc.), or counter payment by showing personal information, registration information, corresponding information of the medicine or examination and test, etc., which are not limited here. After the payment is completed, a guidance note can be generated in the flow-stage of the inquiry for the guidance note. Optionally, the guidance note can provide information for getting medicine (name of the medicine, place for getting the medicine, etc.) and information on items of examination (contents of the examination, department and place of the examination, etc.), so that the target object can conveniently go to corresponding place to complete various required operations according to the guidance note. Optionally, the flow-stage of the inquiry for the guidance note can prompt the target object according to the address, time and order requirements of different operations, so as to facilitate the target object to arrange the time and items of the examination reasonably, and to get medicine and wait in line for the items of the examination accordingly. For example, when the target object is waiting in line for a blood draw, and receives a reminder of B-ultrasound examination at this time, the target object can choose to return to B-ultrasound department first, or choose to continue to complete the blood draw and to wait in line for the B-ultrasound examination again, therefore the visit time of each target object is saved, and the hospital can be facilitated to make reasonable diversion.

Then, in the flow-stage of the display of electronic report, the target object can view the electronic report such as the diagnosis result made by the doctor, a drug list, an examination report of various examinations, a hospitalization report, etc. In addition, in this flow-stage, targeted contents such as advices for a follow-up visit and precautions are also recommended according to the electronic report of the target object.

After the completion of the whole flow of the diagnosis-and-treatment as described above, the target object can further enter the flow-stage of the satisfaction survey according to the visit experience of the whole flow, so as to feedback the satisfaction of the visit and promote the continuous improvement of the hospital and all parties providing services.

Examples of various basic flow information of the diagnosis-and-treatment according to the embodiments of the present disclosure have been described in detail above. In particular applications of the embodiments of the present disclosure, the basic flow information may be different according to the requirements of different target objects, different diseases and different hospital departments, and is not limited here.

In an embodiments of the present disclosure, the determination unit 720 is configured to determine flow progress information. The flow progress information may be different according to the visit flow-stage which the target object visiting a doctor is currently undergoing. For example, when the target object is in the flow-stage of triage, the flow progress information can be, for example, information on symptoms of disease for the target object, etc. When the target object is in the flow-stage of the payment for registration or in the flow-stage of the check-in for visit, the flow progress information can be, for example, whether the payment for registration or the check-in for visit is completed. When the target object is in the flow-stage of the inquiry for guidance note, the flow progress information can be, for example, the completed tasks of the guidance note and the uncompleted tasks of the guidance note for the target object, or specific contents of the examination report with the examination results having been obtained. When the target object has completed all flow-stages of the diagnosis-and-treatment, the flow progress information can enter the flow-stage of the satisfaction survey, etc.

Optionally, as shown in FIG. 7, the determination unit 720 may include an update sub-unit 721, which is configured to acquire the flow progress information updated in real time through a server. The server in the embodiments of the present disclosure may be the aforementioned Hospital Information System (HIS), laboratory information management system (LIS), picture archiving and communication systems (PACS), or contain the contents stored and interacted in these systems. By interacting with the server, the update sub-unit 721 can update the flow progress information in real time, and update the diagnosis-and-treatment flowchart and the state progress of the flow-stages in the diagnosis-and-treatment flowchart accordingly.

The generation unit 730 is configured to generate a diagnosis-and-treatment flowchart and corresponding status indication information according to the basic flow information and the flow progress information, wherein the status indication information indicates a status progress of at least one flow-stage in the diagnosis-and-treatment flowchart.

According to an embodiment of the present disclosure, the generation unit 730 can generate a whole diagnosis-and-treatment flowchart for the target object according to the basic flow information and the flow progress information, and perform a state indication for the state progresses of one or more flow-stages in the whole diagnosis-and-treatment flowchart. Considering factors such as diversity of diseases, the difference between the situations of target objects visiting a doctor, etc. as mentioned above, the whole diagnosis-and-treatment flowchart for different target objects may be significantly different, and the diagnosis-and-treatment flowchart may also be different according to different progresses of the visit of a target object. For example, if the target object is in the stage of the follow-up visit, a certain target department or doctor may have been determined already, and the target object may even visit a doctor directly without registration, in this case, the generation unit 730 may omit the flow-stage of the triage and the flow-stage of the registration, etc. For another example, when the doctor can directly provide diagnosis results after diagnosis and the aforementioned operation of getting medicine or further examination is no longer needed, the diagnosis-and-treatment flow of the target object generated by the generation unit 730 can directly jump from the flow-stage of the check-in for visit to the flow-stage of the display of electronic report as required, without performing the flow-stage of the self-service payment, the flow-stage of the inquiry for guidance note, etc. Or, for example, when the target object has undergone corresponding examinations in the flow-stage of the inquiry for guidance note and needs to register again to check results, the diagnosis-and-treatment flow of the target object generated by the generation unit 730 may be necessary to jump to the flow-stage of the registration again. Therefore, in view of the above-mentioned various personalized situations for the visit, according to the embodiments of the present disclosure, the generation unit 730 can generate the diagnosis-and-treatment flowchart for different target objects, different diseases and different flow progresses, and the status progress can be constantly changed and updated according to actual situations.

Optionally, the status progress of the flow according to the embodiments of the present disclosure can be displayed through different status indication information. For example, in the diagnosis-and-treatment flowchart, the completed flow-stage can be displayed in gray, while the uncompleted flow-stage can be displayed in white. Accordingly, for the completed flow-stage, specific contents of the flow-stage according to the selection of the target object can be provided, while for the uncompleted flow-stage, the specific contents of the flow-stage cannot be displayed, or only some operation prompts of the flow-stage can be displayed.

FIG. 2 illustrates an example of displaying a user interface of a diagnosis-and-treatment flowchart according to an embodiment of the present disclosure. It can be inferred from the diagnosis-and-treatment flowchart shown in FIG. 2 that in this example, the flow-stage of triage, the flow-stage of the registration and the flow-stage of the payment for registration have all been completed, and what is needed to be performed currently is the subsequent flow-stage of the check-in for visit.

Back to FIG. 7, optionally, the acquisition unit (710) can include an inquiry information acquisition sub-unit (711) configured to acquire flow-stage inquiry information. Further, the generation unit (730) further comprises a flow presentation sub-unit (731) configured to present specific contents of a flow-stage in the diagnosis-and-treatment flowchart corresponding to the flow-stage inquiry information according to the flow-stage inquiry information acquired by the inquiry information acquisition sub-unit. Optionally, the flow-stage inquiry information according to the embodiments of the present disclosure can be acquired by selecting a corresponding flow-stage on the user interface by the target object. For example, the flow-stage inquiry information can be obtained by clicking a certain flow-stage in the diagnosis-and-treatment flowchart by the target object. In addition, optionally, specific contents of the flow-stage presented by the flow-stage inquiry information may be different with different flow-stages. For example, the specific contents of the flow-stage of the triage can be triage information, the specific contents of the flow-stage of the registration can be registration information, the specific contents of the flow-stage of the payment for registration can be whether the payment for registration is made, the specific contents of the flow-stage of the check-in for visit can be whether the check-in is made, the specific contents of the flow-stage of the waiting can be waiting order and department information, the specific contents of the flow-stage of the self-service payment can be whether the payment is made, the specific contents of the flow-stage of the inquiry for guidance note can be a guidance note, a drug list, whether an examination is to be made, etc., the specific contents of the flow-stage of the display of the electronic report can be an electronic report, and the specific contents of the flow-stage of the satisfaction survey can be specific options for satisfaction survey. In addition, as mentioned above, for the completed flow-stage, specific contents of the flow-stage can be provided, while for the uncompleted flow-stage, specific contents of the flow-stage cannot be provided, or only operation prompts of the flow-stage can be provided.

In addition, the flow presentation sub-unit (731) can further be configured to present an interactive option for the diagnosis-and-treatment flowchart when the flow presentation sub-unit (731) presents the specific contents of the flow-stage in the diagnosis-and-treatment flowchart, wherein the diagnosis-and-treatment flowchart is presented when the flow presentation sub-unit (731) acquires an interactive instruction through the interactive option. Specifically, after presenting the specific contents of a corresponding flow-stage by obtaining the flow-stage inquiry information, the flow presentation sub-unit 731 can further present the whole diagnosis-and-treatment flowchart on the basis of the specific contents of a certain process through the interactive option for the whole diagnosis-and-treatment flowchart provided in this step. For example, the interactive option can be a sidebar button on the presentation interface of the specific contents of a certain process, and the sidebar button can be fixed or suspended, which is not limited here. The target object can provide an interactive instruction by clicking the sidebar button. After the interactive instruction provided by the target object is acquired, the diagnosis-and-treatment flowchart for the target object can be displayed on the sidebar of the user interface according to the interactive instruction of the target object. Optionally, the diagnosis-and-treatment flowchart presented in the sidebar may also prompt the flow-stage inquiry information when the target object clicks a corresponding flow-stage, and jump to the specific contents of the flow-stage corresponding to the flow-stage inquiry information again according to actual situations, so as to realize the display of the whole flow of the diagnosis-and-treatment and the switching and jumping of the displayed contents among different flow-stages. FIG. 5 illustrates an example of an sidebar interaction option according to an embodiment of the present disclosure. As shown in FIG. 5, on the premise of displaying the specific contents of a certain flow-stage, the diagnosis-and-treatment flowchart of the target object can be displayed on the sidebar for the target object to click to select a corresponding flow-stage.

Optionally, the acquisition unit 710 can further include a history information acquisition sub-unit 712 configured to acquire interaction history information. In addition, the generation unit 730 can include a history presentation sub-unit 732 configured to generate a corresponding historical diagnosis-and-treatment flowchart according to the interaction history information acquired by the history information acquisition sub-unit 712. The historical visit information of the target object provides a very important reference for the triage and visit of the target object. Therefore, in the embodiments of the present disclosure, the history presentation sub-unit 732 may present the historical diagnosis-and-treatment flowchart by obtaining the interaction history information, and display a corresponding electronic report and other contents, as a reference for the target object or the doctor.

The apparatus for guiding the diagnosis-and-treatment according to the embodiments of the present disclosure can realize flow customization and status indication aiming at different diagnosis-and-treatment target objects and different diagnosis-and-treatment flows, so as to improve user experience and improve the efficiency of visit.

The embodiments of the present disclosure also provide an apparatus 800 for guiding a diagnosis-and-treatment, the structural block diagram of which is shown in FIG. 8, and includes a processor 810 and a memory 820. It should be understood that the structure of the apparatus 800 for guiding the diagnosis-and-treatment shown in FIG. 8 is only exemplary and not limited, and the apparatus 800 for guiding the diagnosis-and-treatment may also have other components according to practical application requirements.

In the embodiments of the present disclosure, the processor 810 and the memory 820 can communicate with each other directly or indirectly. Components such as the processor 810 and the memory 820 can communicate through a network connection. The network may include a wireless network, a wired network, and/or any combination of the wireless network and the wired network. The network may include local area network, Internet, telecommunication network, Internet of Things based on the Internet and/or the telecommunication network, and/or any combination of the above networks. The wired network can utilize transmission twisted pair, coaxial cable, optical fiber, etc. for communication, and the wireless network can utilize 3G/4G/5G mobile communication network, Bluetooth, Zigbee, WiFi, etc. for communication. The disclosure does not limit the type and function of the network here.

The processor 810 may control other components in the display apparatus 600 to perform desired functions. The processor 810 may be a central processing unit (CPU), a tensor processor (TPU), a graphics processor (GPU), and other devices with data processing capabilities and/or program execution capabilities. A central processing unit (CPU) can be X86 or ARM architecture. The GPU can be directly integrated into a main board or built in the north bridge chip of the main board. The GPU can also be built into the central processing unit (CPU).

The memory 820 may include any combination of one or more computer program products, which may include various forms of computer-readable storage media, such as volatile memory and/or nonvolatile memory. The volatile memory may include, for example, a random access memory (RAM) and/or cache, etc. The nonvolatile memory may include, for example, read-only memory (ROM), hard disk, erasable programmable read-only memory (EPROM), portable compact disk read-only memory (CD-ROM), USB memory, flash memory, and the like.

One or more computer readable codes or instructions can be stored in the memory 820, and the processor 610 can execute the computer instructions to perform the method for guiding diagnosis-and-treatment, and the method includes: acquiring basic process information of the diagnosis-and-treatment; determining flow progress information; and generating a diagnosis-and-treatment flowchart and corresponding status indication information according to the basic flow information and the flow progress information, wherein the status indication information indicates a status progress of at least one flow-stage in the diagnosis-and-treatment flowchart. For a detailed description of the method, please refer to the relevant description of the method in this specification, and it will not be repeated here. Various applications and various data can also be stored in the computer-readable storage medium.

According to another aspect of the present disclosure, there is also provided a computer-readable storage medium on which computer instructions are stored, wherein when the computer instructions are executed on a processor, the processor is caused to perform the following steps: acquiring basic process information of the diagnosis-and-treatment; determining flow progress information; and generating a diagnosis-and-treatment flowchart and corresponding status indication information according to the basic flow information and the flow progress information, wherein the status indication information indicates a status progress of at least one flow-stage in the diagnosis-and-treatment flowchart.

A computer-readable medium may have many forms, including tangible storage medium, carrier wave medium or physical transmission medium. Stable storage media can include optical disks or magnetic disks, and storage systems used in other computers or similar devices, which can realize the system components described in the Figures. Unstable storage media can include dynamic memories, such as the main memory of a computer platform. Tangible transmission media can include coaxial cables, copper cables, and optical fibers, such as lines forming buses in computer systems. Carrier wave transmission media can transmit electrical signals, electromagnetic signals, sound waves or light waves. These signals can be generated by radio frequency or infrared data communication. Common computer-readable media include: hard disks, floppy disks, magnetic tapes, and any other magnetic media, CD-ROM, DVD, DVD-ROM, any other optical medium; punch cards, any other physical storage media containing pinhole patterns; RAM, PROM, EPROM, FLASH-EPROM, any other memory chips or tapes; carrier waves for transmitting data or instructions, cables or connecting apparatuses for transmitting carrier waves, and any other program codes and/or data that can be read by a computer. Many of these forms of computer-readable media appear in the process of executing instructions and delivering one or more results by a processor.

According to still another aspect of the present disclosure, there is also provided a computer, which may include the apparatus for guiding a diagnosis-and-treatment as described above. Specifically, the computer can be implemented as one or more special or general computer system modules or components, such as personal computers, notebook computers, tablet computers, mobile phones, personal digital assistance (PDA), smart glasses, smart watches, smart rings, smart helmets and any smart portable devices.

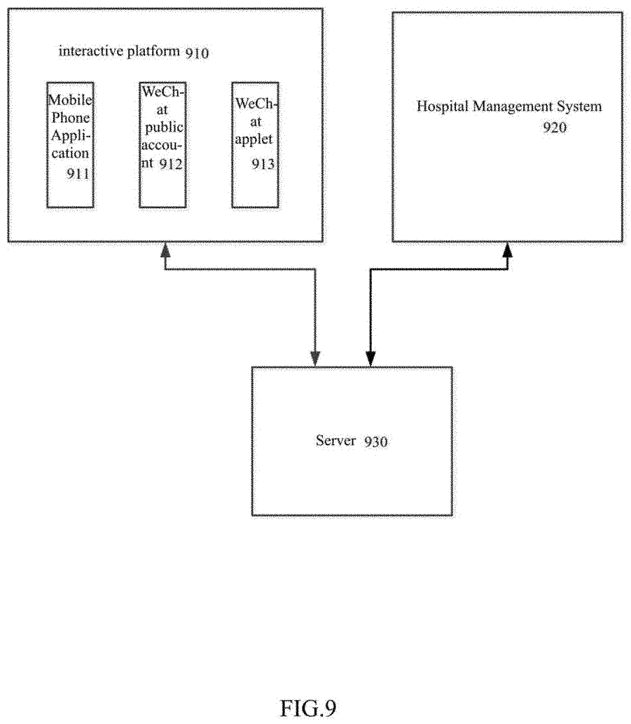

Next, a system for guiding a diagnosis-and-treatment according to an embodiment of the present disclosure will be described with reference to FIG. 9. FIG. 9 illustrates a structural block diagram of an example of a system for guiding diagnosis-and-treatment according to an embodiment of the present disclosure. As shown in FIG. 9, the system 900 for guiding the diagnosis-and-treatment includes: an interactive platform 910 configured to execute the method for guiding the diagnosis-and-treatment as previously described; a hospital management system 920; and a server 930, wherein the server communicates and interacts with the interactive platform and the hospital management system respectively. Specifically, the interactive platform in the embodiments of the present disclosure can be a mobile platform, which can be a mobile device, such as a notebook computer, a mobile phone, a tablet computer, a personal digital assistant, smart glasses, a smart watch, a smart ring, a smart helmet and any smart portable device. The interactive platform 910 can implement the method for guiding the diagnosis-and-treatment as defined in the embodiments of the present disclosure through carriers loaded therein, such as mobile phone application 911, WeChat public account 912, and WeChat applet 913. The hospital management system 920 may include the aforementioned hospital information system, laboratory information management system, picture archiving and communication systems, a transmission system and doctor workstation, etc., and is used for managing and storing the information, examination reports, payment status, etc. of target objects undergoing diagnosis-and-treatment in the hospital. The server 930 can be used as a database to store the data provided by the interactive platform 910 and the hospital management system 920, and interact information and data with the interactive platform 910 and the hospital management system 920 respectively.

According to the method, apparatus, system for guiding the diagnosis-and-treatment, computer-readable storage medium and computer disclosed above, a diagnosis-and-treatment flowchart showing the whole flow of the diagnosis-and-treatment can be generated according to the basic diagnosis-and-treatment flow information of the diagnosis-and-treatment and the flow progress information of the target object, and state indication information indicating the state progress of the flow in the diagnosis-and-treatment flowchart can be generated. According to the method, apparatus, system for guiding the diagnosis-and-treatment guidance, and computer readable storage medium disclosed by the present disclosure, flow customization and status indication aiming at different diagnosis-and-treatment target objects and different diagnosis-and-treatment flows can be realized, so as to improve user experience and improve the efficiency of visit.

Unless otherwise defined, all terms (including technical and scientific terms) used herein have the same meanings as commonly understood by those of ordinary skill in the art to which this disclosure belongs. It should also be understood that terms such as those defined in general dictionaries should be interpreted as having meanings consistent with their meanings in the context of the related art, and should not be interpreted in an idealized or extremely formal sense unless explicitly defined herein.

The above is a description of the present disclosure, and should not be considered as a limitation thereof. Although several exemplary embodiments of the present disclosure have been described, those skilled in the art will readily understand that many modifications can be made to the exemplary embodiments without departing from the novel teaching and advantages of the present disclosure. Therefore, all such modifications are intended to be included within the scope of the present disclosure as defined by the claims. It should be understood that the above is a description of the present disclosure, and should not be regarded as limited to the specific embodiments disclosed, and modifications to the disclosed embodiments and other embodiments are intended to be included within the scope of the appended claims. This disclosure is defined by the claims and their equivalents.

1-5. (canceled)

6. A method for guiding diagnosis-and-treatment, comprising:
acquiring basic flow information of the diagnosis-and-treatment;
determining flow progress information;
generating a diagnosis-and-treatment flowchart and corresponding status indication information according to the basic flow information and the flow progress information, wherein the status indication information indicates a status progress of at least one flow-stage in the diagnosis-and-treatment flowchart.

7. The method of claim 6, wherein the method further comprises:
acquiring flow-stage inquiry information;
presenting, according to the flow-stage inquiry information, specific contents of a flow-stage in the diagnosis-and-treatment flowchart corresponding to the flow-stage inquiry information.

8. The method of claim 7, wherein the method further comprises:
further presenting an interactive option for the diagnosis-and-treatment flowchart when the specific contents of the flow-stage in the diagnosis-and-treatment flowchart is presented, wherein the diagnosis-and-treatment flowchart is presented when an interactive instruction is acquired through the interactive option.

9. The method according to claim 6, wherein the method further comprises:
acquiring interaction history information; and
generating a corresponding historical diagnosis-and-treatment flowchart according to the interaction history information.

10. The method according to claim 6, wherein determining the flow progress information further comprises:
acquiring the flow progress information updated in real time through a server.

11. An apparatus for guiding diagnosis-and-treatment, comprising:
one or more processors;
one or more memories,
wherein the memories store computer readable codes which, when executed by the one or more processors, perform the following steps:
acquiring basic flow information of the diagnosis-and-treatment;
determining flow progress information;
generating a diagnosis-and-treatment flowchart and corresponding status indication information according to the basic flow information and the flow progress information, wherein the status indication information indicates a status progress of at least one flow-stage in the diagnosis-and-treatment flowchart.

12. A computer-readable storage medium on which computer instructions are stored, wherein when the computer instructions are executed on a processor, the processor is caused to perform the steps of:
acquiring basic process information of the diagnosis-and-treatment;
determining flow progress information; and
generating a diagnosis-and-treatment flowchart and corresponding status indication information according to the basic flow information and the flow progress information, wherein the status indication information indicates a status progress of at least one flow-stage in the diagnosis-and-treatment flowchart.

13. A system for guiding diagnosis-and-treatment, comprising:
an interactive platform configured to execute the method for guiding the diagnosis-and-treatment according to claim 6;
a hospital management system; and
a server, wherein the server communicates and interacts with the interactive platform and the hospital management system respectively.

14. The system according to claim 13, wherein,
the hospital management system is configured to manage and store information, an examination report, a payment status of a target object undergoing the diagnosis-and-treatment in the hospital, and
the server is used as a database to store the data provided by the interactive platform and the hospital management system, and is configured to interact information and data with the interactive platform and the hospital management system respectively.

15. The system according to claim 13, wherein, the interactive platform is further configured to:
acquire flow-stage inquiry information;
present, according to the flow-stage inquiry information, specific contents of a flow-stage in the diagnosis-and-treatment flowchart corresponding to the flow-stage inquiry information.

16. The system according to claim 15, wherein, the interactive platform is further configured to:
further present an interactive option for the diagnosis-and-treatment flowchart when the specific contents of the flow-stage in the diagnosis-and-treatment flowchart is presented, wherein the diagnosis-and-treatment flowchart is presented when an interactive instruction is acquired through the interactive option

17. The system according to claim 13, wherein, the interactive platform is further configured to:
acquire interaction history information; and
generate a corresponding historical diagnosis-and-treatment flowchart according to the interaction history information.

18. The method according to claim 7, wherein the method further comprises:
acquiring interaction history information; and
generating a corresponding historical diagnosis-and-treatment flowchart according to the interaction history information.

19. The method according to claim 8, wherein the method further comprises:
acquiring interaction history information; and
generating a corresponding historical diagnosis-and-treatment flowchart according to the interaction history information.

20. The method according to claim 7, wherein determining the flow progress information further comprises:
acquiring the flow progress information updated in real time through a server.

21. The method according to claim 8, wherein determining the flow progress information further comprises:
acquiring the flow progress information updated in real time through a server.

22. The apparatus of claim 11, wherein the memories store computer readable code which, when executed by the one or more processors, further perform the following steps:
acquiring flow inquiry information;
presenting specific contents of a flow-stage in the diagnosis-and-treatment flowchart corresponding to the flow inquiry information according to the flow inquiry information.

23. The apparatus of claim 22, wherein the memories store computer readable code which, when executed by the one or more processors, further perform the following step:
further presenting an interactive option for the diagnosis-and-treatment flowchart when the specific contents of the flow-stage in the diagnosis-and-treatment flowchart is presented, wherein the diagnosis-and-treatment flowchart is presented when an interactive instruction is acquired through the interactive option.

24. The apparatus of claim 11, wherein the memories store computer readable code which, when executed by the one or more processors, further perform the following steps:
acquiring interaction history information; and
generating a corresponding historical diagnosis-and-treatment flowchart according to the interaction history information.

25. The apparatus of claim 11, wherein determining the flow progress information further comprises:
acquiring the flow progress information updated in real time through a server.
